(19) United States
(12) Patent Application Publication
(10) Pub. No.: US 2009/0306148 A1
(43) Pub. Date: Dec. 10, 2009

(54) Use of Thiazolidinediones for the Partial Inhibition of Androgen Binding to Aromatase

(76) Inventors: Donna Lin Tang Seto-Young, New York, NY (US); Leonid Poretsky, New York, NY (US)

Correspondence Address: OSTRAGER CHONG FLAHERTY & BROITMAN PC, 570 LEXINGTON AVENUE, FLOOR 17, NEW YORK, NY, 10022-6894, US

(21) Appl. No.: 12/463,522

(22) Filed: May 11, 2009

Related U.S. Application Data
(60) Provisional application No. 61/052,191, filed on May 10, 2008.
(60) Provisional application No. 61/061,570, filed on Jun. 13, 2008.

(51) Int. Cl. A61K 31/4439

(52) U.S. Cl. 514/342

(57) ABSTRACT

A method for the treatment or prophylaxis of a disorder, wherein the disorder is affected by estrogen, comprising partially inhibiting aromatase activity or interfering with the binding of androgen to aromatase by administering a therapeutically effective amount of a thiazolidinedione to the patient. The use of rosiglitazone or pioglitazone to partially inhibit the binding of androgen to aromatase by at least 20% is preferred.

### RELATED APPLICATIONS

This application claims the priority of both U.S. provisional patent application Ser. No. 61/052,191 filed May 10, 2008 and U.S. provisional patent application Ser. No. 61/061,570 filed Jun. 13, 2008, the entire contents of which are being incorporated herein by reference.

### FIELD OF THE INVENTION

The present invention relates to the use of thiazolidinediones (“TZDs”), in particular rosiglitazone or piglitazone, to inhibit or interfere with the binding of androgens to aromatase, in particular the use of TZDs to partially and reversibly inhibit or interfere with this binding. The present invention further relates to the use of partial inhibition of androgen binding to aromatase for the prophylaxis and treatment of hormone-dependent diseases. The present invention also relates to pharmaceutical compositions and dosage forms comprising thiazolidinediones for the prophylaxis and treatment of hormone-dependent diseases.

### BACKGROUND OF THE INVENTION

Increased exposure to estrogens in the breast is an important risk factor in the genesis and growth of breast cancer and other estrogen-dependent diseases, including gynecomastia, uterine fibroids and endometrial cancer (O. Karaer et al., Acta. Obstet. Gynecol. Scand. 83:699-707, 2004; M. Salhab et al., 99 Breast Cancer Res. 99:155-162, 2006). Estrogens are derived from androgens through the action of aromatase, a component of the cytochrome P450 enzyme complex that catalyzes the hydroxylation of androstenedione to estrone and of testosterone to estradiol. Particularly in cases of breast cancer, high tumor levels of aromatase mRNA expression correlate with poor survival of patients (M. Salhab et al., 99 Breast Cancer Res. 99:155-162, 2006).

Presently, two classes of pharmacologic agents are used in the treatment of estrogen receptor-positive breast cancer: the selective estrogen receptor modulators, such as tamoxifen, which block the interaction of estrogen with its specific receptor, and aromatase inhibitors (AIs), which suppress the biosynthesis of estrogen by blocking the conversion of androgens to estrogens (K. Altundag et al., The Oncologist 11:553-562, 2006; I. E. Messinis, Human Reproduction, 20:2688-2697, No. 10, 2005). Recently, aromatase inhibitors used as adjuvant therapy, were shown to be equivalent or superior to tamoxifen as the endocrine therapy for postmenopausal patients with breast cancer (K. Altundag. et al., The Oncologist 11:553-562, 2006). Attempts to reduce rather than eliminate estradiol with aromatase inhibitors, such as letrozole and fadrozole, have shown marked intersubject heterogeneity leading A. Kendall and M. Dowsett in Endocrine-Related Cancer 13: 827-837, 2006 to suggest that the “use of low-dose AI might require individual titration with consequent feasibility issues.”

Thiazolidinediones (TZDs, troglitazone, rosiglitazone and pioglitazone) are peroxisome proliferator activated receptor-μ (PPAR-μ) agonists. It has been proposed that TZDs can be used as therapeutic agents for women with polycystic ovary syndrome (PCOS) since they may improve ovulatory rates and reduce androgen levels (S. Dronavalli et al., Clin. Obstetr. Gynecol. 50: 244-254, 2007; N. Brettenthaler et al., J. Clin. Endocrinol. Metab. 89:3835-3840, 2004; V. Sepilian et al., J. Clin. Endocrinol. Metab. 90: 60-65, 2005; R. S. Legro et al., Am. J. Obstet. Gynecol. 196: 402 e1-10, discussion 402 e10-1, 2007). Although these effects have been attributed to systemic insulin-sensitizing effects of TZDs and consequent reduction of hyperinsulinemia, TZDs also directly affect androgen and estrogen production in human ovarian cell culture (D. Seto-Young et al., J. Clin. Endocrinol. Metab. 90:6099-6105, 2005). Conflicting reports have been published regarding the effects of TZDs on the expression and activity of aromatase in the ovary. S. Gasic et al. reported the absence of TZD effects on aromatase in porcine granulosa cells. (S. Gasic et al., Endocrinology 139:4962-4966, 1998), while other studies reported TZD-induced suppression of enzymatic expression and activity of aromatase in human granulosa and granulosa cell carcinoma cell lines (W. Q. Fan et al., Endocrinology 146:85-92, 2005; Y. M. Mu et al., Biochem. Biophys. Res. Commun. 271:710-713). Other previous studies suggested that TZDs inhibited aromatase activity in mixed human ovarian cell culture containing stromal, thecal and granulosa cells (D. Seto-Young et al., J. Clin. Endocrinol. Metab. 90: 6099-6105, 2005).

PPAR-γ ligands have a potent antiproliferative activity against a variety of neoplastic cells, including inhibition of the proliferation of human breast cancer cells. PPAR-γ agonists have been shown to induce degradation of estrogen receptor-α in breast cancer cells and uterine leiomyoma (C. Qui et al., Cancer Res. 68:958-964, 2003; K. D. Houston et al., Cancer Res. 63:1221-1227, 2003). Further, PPAR-γ agonist 15-deoxy-δ^{12, 14}-prostaglandin J₂ inhibits transcriptional activity of estrogen receptor-α via covalent modification of DNA-binding (H. J. Kim et al., Cancer Res. 67:2595-2602, 2007) and induces apoptosis and inhibition of breast cancer cell growth (K. Kondoh et al., 313 Exp. Cell Res. 313:3486-3497, 2007). Recently, TZDs were shown to promote breast cancer cell apoptosis induced by tumor necrosis factor-related apoptosis-inducing ligand (TRAIL) suggesting that combination of TZDs and TRAIL may be an effective therapy for breast cancer (M. Lu et al., J. Biol. Chem. 280:6742-6751, 2005). The role of PPAR-γ ligands in chemoprevention of breast cancer in carcinogen nitrosoethylurea (NMU)-fed animals has been also shown (N. Suh et al., Cancer Res. 59:5671-5673, 1999).

Studies have shown that exposure of mesenchymal stem cells derived from bone marrow to TZDs potentiated their adipogenic, rather than osteogenic, differentiation (S. Benvenuti et al., 30 Endocrinol. Invest. 30:26-30, 2007). This effect was manifested by the appearance of lipid droplets and reduced expression of Runx2 (a marker of osteoclastogenesis) in these cells. These results confirm other studies suggesting that TZDs increase bone marrow adiposity and decrease osteoclastogenesis (S. Benvenuti et al., Endocrinol. Invest. 30:26-30, 2007; S. O. Rzonca et al., 145 Endocrinology 401-406, 2004). For example, TZDs inhibited in vitro bone nodule formation and mineralization (T. Johnson et al., Endocrinology 140:3245-3254, 1999). The analysis of the 4 year data from the Health, Aging and Body Composition (Health ABC) study in older women demonstrated that each year of TZD use was associated with whole body bone mass loss of 0.67% (A. V. Schwartz et al., J. Clin. Endocrinol. Metab. 91:3349-3354, 2006). A number of other studies have suggested that the use of TZDs is associated with a reduction in bone density (A. V. Schwartz et al., J. Clin. Endocrinol. Metab. 91:3349-3354, 2006; A. Grey et al., J. Clin. Endocrinol. Metab. 92:1305-1310, 2007).

### SUMMARY OF THE INVENTION

The use of TZDs in the place of currently used aromatase inhibitors may allow for fewer side effects. Fewer side effects may permit the administration of TZDs for a longer period of time than is recommended for non-TZD aromatase inhibitors currently used for the prevention of breast cancer recurrence. It may be safe to use TZDs for at least a year, preferably for three to five years. In a particular preferred embodiment of the invention, TZDs could be used for the lifetime of the patient. The lifetime use of TZDs to treat diabetes establishes the safety of TZDs for other long term uses.

Furthermore, the inhibition, particularly the partial and reversible inhibition of the binding of androgens to aromatase, may permit the use of TZDs for prophylaxis and for treatment of conditions and diseases where the reduction or regulation of sex hormones, in particular, estrogen, is desirable. In the following discussion, “conditions” include diseases, abnormalities, and treatments undertaken taken to optimize or improve health and fertility. The partial inhibition of aromatase, interference with the binding of androgens with aromatase and/or the partial reduction of estrogen production, rather than a significant (more than 50%) or complete blockage of estrogen production, could be desirable in treating a number of conditions. Allowing some estrogen production may improve the comfort and health of the patient. These conditions may include the prevention and treatment of pre- and postmenopausal breast cancer and other estrogen-dependent malignancies, endometriosis, gynecomastia (including adolescent gynecomastia), uterine fibroids, premature puberty, hyperestrogenism, male infertility among other conditions which substantially originate or are influenced by the presence of hormone receptors and/or hormone-dependent pathways. These conditions and malignancies also afflict non-human mammals. Furthermore, captive birds and reptiles can suffer various breeding and reproductive disorders which can develop due to excessive exposure to estrogen.

A particular preferred range of the inhibition of the binding of androgen to aromatase is from about 20% to about 38%. When rosiglitazone is used to treat human patients, 2.0 mg to 8.0 mg of rosiglitazone on a daily basis may be a preferred dosage, with a particularly preferred range of 2.0 to 4.0 mg. When pioglitazone is used to treat human patients, 5.0 mg to 45.0 mg of pioglitazone on a daily basis may be a preferred dosage, with a particularly preferred range of 5.0 to 15.0 mg. 4.0 to 8.0 mg of rosiglitazone and 15.0 to 45.0 mg of pioglitazone have a proven safety record in treating diabetes. Other TZDs besides rosiglitazone and pioglitazone may also be suitable such as the TZDs described in U.S. Pat. Nos. 4,287,200, 4,687,777, 5,952,356, 5,965,584, 6,150,383, 6,150,384, 6,166,042, 6,166,043, 6,172,090, 6,211,205, 6,271,243, 6,303,640, 6,329,404, 5,002,953, 5,741,803, 6,288,095; the disclosures of the aforementioned patents are incorporated herein by reference in their entirety.

### BRIEF DESCRIPTION OF THE FIGURES

FIG. 1 shows the effects of TZDs and aromatase inhibitor I on estrone production in the presence of androstenedione as a substrate.

FIG. 2 shows the effects is TZDs and aromatase inhibitor I on estradiol production in the presence of testosterone.

FIG. 3 shows the effects of TZDs on aromatase mRNA and protein expression in the presence of androstenedione or testosterone and various concentrations of insulin, with or without rosiglitazone or pioglitazone.

FIG. 4 shows the effects of TZDs on ¹²⁵[I]-androstenedione and ¹²⁵ [I] testosterone binding to aromatase enzyme.

FIG. 5 shows the in-vivo effects of TZDs on estrogen production in women with PCOS. TZDs reduce circulating insulin levels through their insulin-sensitizing action, which in turn leads to a reduction of circulating testosterone levels.

FIG. 6 shows the effects of TZDs on the Kₘ and Vₘₐₓ of aromatase in the presence of testosterone.

FIG. 7 shows the effects of TZDs on the Kₘ and Vₘₐₓ of aromatase in the presence of androstenedione.

### DETAILED DESCRIPTION OF THE EMBODIMENTS

Embodiments of the present invention will be described with reference to the accompanying figures.

### Materials and Methods

Granulosa Cell Culture

Human granulosa cells were obtained during in vitro fertilization over the course of 18 months and were pooled from several patients at a time to ensure adequate cell number for the experiments. The diagnoses included male factor, tubal factor and uterine factor infertility, endometriosis and anovulation.

Granulosa cells were purified on Percoll gradients and cultured as described in D. Seto-Young et al., J. Clin. Endocrinol. Metab., 88:3385-3391, 2003, which is incorporated herein by reference in its entirety. Purified granulosa cells were resuspended in M199 medium supplemented with 10% heat-inactivated fetal bovine serum (FBS), 10 μg/ml gentamicin and 250 μg/ml amphotericin (Invitrogen Corp., Carlsbad, Calif.). 1 ml of 0.5×10⁵ cells/ml suspension was used for the steroid hormone experiments and 2 ml of 2 to 3×10⁵ cells/ml were used for immunoblot, RT-PCR and binding of androgens to aromatase studies. The cells were incubated for 48 h in 5% carbon dioxide and 90% humidity and then for additional 24 h in the same medium containing 2% FBS. The cells were then incubated for 18 h in the same medium with or without substrates (3 μM androstenedione or testosterone), in the presence or absence of insulin (0-10⁴ ng/ml), with or without rosiglitazone or pioglitazone (25 μM), and with or without 100 or 200 nM aromatase inhibitor I [4-(imidazolylmethyl)-1-nitro-9H-9-xanthenone] (EMB BioSciences, Inc. La Jolla, Calif.).

Radioimmunoassay and Immuno-Sorbent Assay

Estrone and estradiol concentrations in the tissue culture medium were measured using enzyme-linked immuno-sorbent assay (ELISA) (Alpco Diagnostics, Salem, N.H.) or radioimmunoassay (RIA) (Diagnostic Systems Laboratories, Webster, Tex.).

Aromatase Enzyme Expression Using Immunoprecipitation and Immunoblotting Procedure

Rabbit polyclonal anti-human aromatase antibodies (1.4 μg) (Abcam, Inc. Cambridge, Mass.) were added to 1 ml of cell lysate. The lysate buffer contained 20 mM Tris-HCl (pH 7.4), 150 mM NaCl, 1 mM CaCl₂, 1 mM MgCl₂, 1 mM Na orthovanadate, 1% NP40, 1 mM AEBSF (4-2-aminoethyl-benzenesulfonylfluoride, hydrochloride), 0.8 μM aprotinin, 50 μM bestain, 15 μM E-64, 20 μM leupeptin, 10 μM pepstatin A, 1 mM phenylmethylsulfonyl fluoride and 10 μg/ml trypsin inhibitor. The lysate was centrifuged at 6,000 rpm for 3 min and the supernatant was transferred into another micro-centrifuge tube. Immunoprecipitation procedures were modified as described in D. Seto-Young et al., J. Clin. Endocrinol. Metab. 92: 2232-2239, 2007, which is incorporated herein by reference. The mixture was incubated for 3-4 h at 4° C. After Protein A-agarose was added, the mixture was incubated again overnight at 4° C. The immunoprecipitate complexes were collected by centrifugation and washed. The samples were then resuspended in 2× Laemmli buffer (BioRad, Hercules, Calif.) supplemented with 1 mM DTT (Cleland's reagent, 1,4-dithiothreitol, threo-1,4-dimercapto-2,3-butanediol, Roche Inc, Indianapolis, Ind.). Electrophoresis and immunoblotting procedures were performed as described in the manufacturer's recommendations (BioRad, Hercules, Calif.). Mouse anti-human aromatase antibodies ( 1/100 dilution, as suggested in the manufacturer's recommendations, abD Serote, Inc, Kingston, N.H.) were used for probing the expressed aromatase which was transferred to the nitrocellulose paper. The bands were detected by chemiluminescence (Pierce, Rockford, Ill.). For the analysis of the expression of aromatase, NIH Scion Imaging program was used to evaluate integrated band intensity.

Total RNA Isolation and RT-PCR

The total RNA was isolated using the RNAqueous-4PCR kit. The isolation procedure was performed as described in the manufacturer's recommendations (Ambion Inc., Austin, Tex.). The concentration of RNA was determined by reading the absorbance in spectrophotometer at 260 nm and 280 nm.

GeneAmp EZ rTth RNA PCR kit was used for RT-PCR reaction. Thermostable recombinant DNA polymerase was used as both a reverse transcriptase and as a DNA polymerase in a single PCR reaction. The aromatase forward primer was 5′-ACC CTT CTG CGT CGT GTC A-3′ identified as SEQ ID No:1 and the reverse primer was 5′-GAA CTT CTA TGG CAT CTT TCA AAT CC-3′ identified as SEQ ID No: 2. 1 μg purified total RNA and positive control pAW109 RNA were used. The concentrations of the primers, deoxyribose nucleotides, and rTth DNA polymerase were used as described in the manufacturer's recommendations (Applied Biosystems, Branchburg, N.J.). The reverse transcription was performed by incubating the reaction mixture at 42° C. for 5 min, ramping to 65° C. over 5 min (50° C. for 1 min, 55° C. for 1 min, 60° C. for 1 min, 65° C. for 1 min) and 65° C. for 40 min in the GeneAmp PCR System 9700 (Applied Biosystem, Branchburg, N.J.). The PCR reaction was carried out at 94° C. for 1 min, followed by 40 cycles of a two temperature PCR (94° C. for 15 sec and 62° C. for 40 sec), ending with 60° C. for 7 min and then on hold at 4° C.

The cDNA was separated on 2.5% agarose gel in TBE buffer (89 mM Tris, pH 8.3, 89 mM boric acid and 2 mM EDTA). The agarose gel was stained with 2 μg/ml ethidium bromide and DNA bands were visualized on the UV transilluminator.

Purification of PCR DNA Fragments

The RT-PCR generated a 208 bp fragment whose sequence began at 313 bp and ended at 510 bp of aromatase gene. This cDNA fragment contained a Bam HI restriction site at 362 bp. The 208 bp cDNA fragments were eluted from the agarose gel by cutting a well below the 208 bp position. 3×TBE buffer was placed into the well and then electrophoresed for 5 min to 10 min until the 208 bp cDNA fragments were eluted into the well. The cDNA fragments were collected and 2 volumes of 100% ethanol, 0.1 volume of 5 M ammonium acetate and 0.02 volume of linear acrylamide were added. The mixture was placed at −20° C. for 2 h. The precipitated cDNA was incubated with 2 units of Bam HI restriction enzyme for 2 h at 37° C. One volume of DNA loading buffer was added and the mixture was then electrophoresed on 3% agarose gel in TBE buffer. The restriction cDNA map was examined under the UV transilluminator. A 148 bp DNA fragment was observed, proving that RT-PCR generated fragments were in the position corresponding to that of aromatase gene.

Androstenedione and Testosterone Binding Experiments

The aromatase enzyme was immunoprecipitated as described above. Purified aromatase bound to Protein A-agarose was added to 250 μl of solution containing the final concentration of 1 μM testosterone (2 μCi of [1251]/ml testosterone with the specific radioactivity of 857 μCi/nmol) or androstenedione (3.5 μCi of [1251]/ml androstenedione with the specific radioactivity of 430 μCi/nmol), with insulin (0, 10, 10², 10³ ng/ml) and with or without 25 μM rosiglitazone or pioglitazone. The mixture was incubated at room temperature for 30 min and then centrifuged at 6000 rpm for 3 min. The pellets were washed two times with 400 μl Dulbecco's phosphate saline. The bound testosterone or androstenedione, expressed as % of total testosterone or androstenedione, was measured in Wizard gamma counter.

Statistical Analysis

Two-way analysis of variance (ANOVA) was used to compare mean values according to insulin concentrations in the presence or absence of rosiglitazone or pioglitazone, and with or without aromatase inhibitor I. The statistical interactions between the sets of data obtained with or without rosiglitazone or pioglitazone and with or without aromatase inhibitor I were examined. Pairwise Bonferroni-adjusted contrasts were used to determine statistical significance. Analysis of covariance (ANCOVA) was used to assess the statistical significance of mean differences among the sets of data according to insulin concentrations in the presence or absence of rosiglitazone or pioglitazone and with or without aromatase inhibitor I. Adjustments were made for initial inhibition of estradiol or estrone production induced by rosiglitazone or pioglitazone in the absence of insulin, with or without aromatase inhibitor I.

Results

In the absence of a substrate, the purified granulosa cells produced approximately 500 ng/ml of estrone and 1000 ng/ml estradiol. Adding 3 μM androstenedione or testosterone as a substrate to the tissue culture medium resulted in 20-25 fold increase of estrone and estradiol production.

Effects of TZDs on Estrone Production in the Presence of Androstenedione (3 μM)

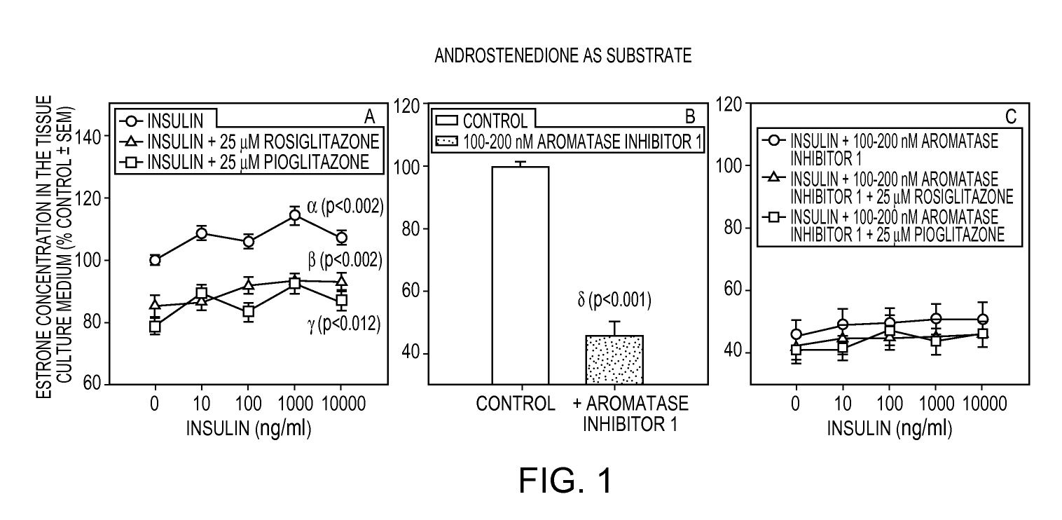

In the absence of aromatase inhibitor I, insulin stimulated estrone production by 114% (p<0.002) while rosiglitazone or pioglitazone inhibited estrone production by 22% (p<0.002 for rosiglitazone and p<0.012 for pioglitazone, respectively) (FIG. 1A; α, p<0.002, n=17, compared to baseline (absence of insulin, rosiglitazone or pioglitazone); β, p<0.002, n=14 and γ, p<0.012, n=14, compared to control (in the presence of insulin and in the absence of rosiglitazone or pioglitazone)).

In the presence of aromatase inhibitor (100 to 200 nM), estrone production was inhibited by 55% (FIG. 1B; δ, p<0.001, n=8). In the presence of aromatase inhibitor I and insulin, inhibitory effect of rosiglitazone or pioglitazone on insulin-induced estrone production was completely abolished (FIG. 1C).

Effects of TZDs on Estradiol Production in the Presence of Testosterone (3 μM)

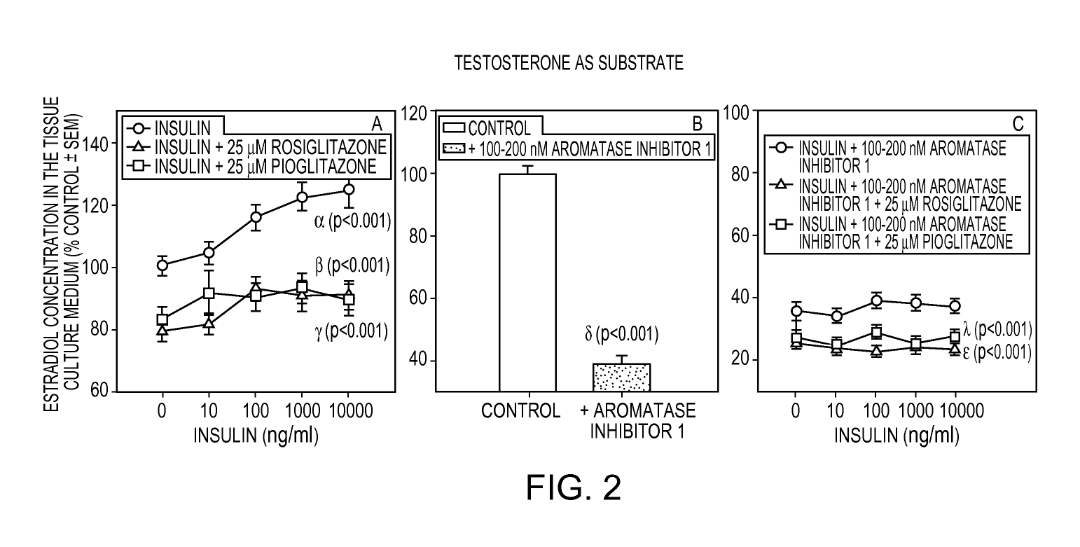

In the absence of aromatase inhibitor I, insulin stimulated estradiol production by 124% (p<0.001) (FIG. 2A) while rosiglitazone or pioglitazone inhibited estradiol production by 34% (p<0.001) (FIG. 2A; α, p<0.001, n=12, compared to baseline (absence of insulin, rosiglitazone or pioglitazone); β, p<0.001, n=12 and γ, p<0.001, n=12, compared to control (presence of insulin and absence of rosiglitazone or pioglitazone)).

In the presence of aromatase inhibitor I, estradiol production was inhibited by 72% (FIG. 2B; δ, p<0.001, n=5) and insulin-induced stimulation of estradiol production was completely abolished (FIG. 2C). In the presence of aromatase inhibitor I and insulin, inhibitory effects of rosiglitazone and pioglitazone on insulin-induced estradiol production were reduced by 62% (ε, p<0.001 and λ, p<0.001, compared to control (presence of insulin and aromatase inhibitor I and absence of rosiglitazone or pioglitazone); compare FIG. 2A to FIG. 2C).

Effects of TZDs on Aromatase Expression

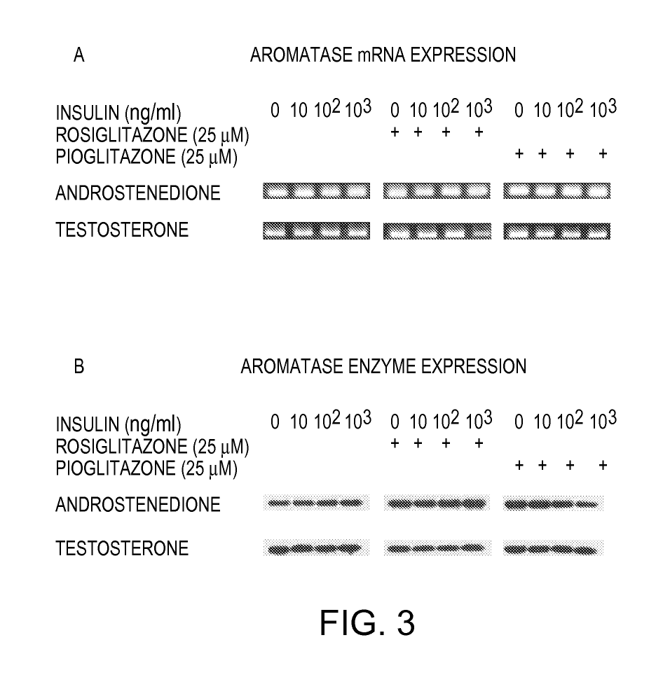

FIG. 3A shows representative RT-PCR analysis of aromatase mRNA expression in the presence of androstenedione or testosterone and various concentrations of insulin, with or without rosiglitazone or pioglitazone. When androstenedione or testosterone was used as substrate, insulin alone had no effect on aromatase mRNA (FIG. 3A). Addition of 25 μM rosiglitazone or pioglitazone had no significant effect on aromatase mRNA compared to control either in the absence or in the presence of insulin (FIG. 3A).

FIG. 3B is a representative immunoblot analysis or aromatase expression in the presence of androstenedione or testosterone, with various concentrations of insulin, with or without rosiglitazone or pioglitazone. When androstenedione or testosterone was used as substrate, insulin alone did not affect aromatase enzyme (protein) expression (FIG. 3B). Addition of 25 μM rosiglitazone or pioglitazone had no significant effect on aromatase enzyme expression compared to control, either in the absence or in the presence of insulin (FIG. 3B).

Effect on TZDs on Androgen Binding to the Aromatase Enzyme

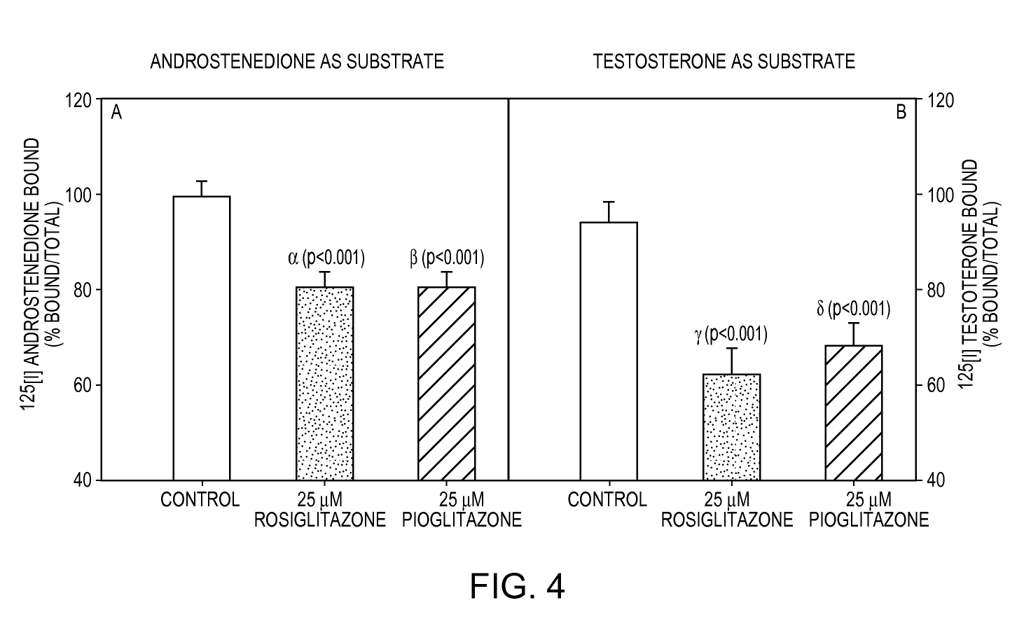

Insulin alone had no effect on the binding of androstenedione or testosterone to aromatase. In the presence of 25 μM rosiglitazone or pioglitazone, ¹²⁵I-androstenedione binding to the aromatase enzyme was inhibited by 20% (FIG. 4A; α, p<0.001, n=5, β, p<0.001, n=5 compared to control (absence of rosiglitazone or pioglitazone)). Similarly, rosiglitazone inhibited ¹²⁵I-testosterone binding to the aromatase enzyme by 38% (p<0.001) (FIG. 4B) and pioglitazone by 32% (p<0.001) (FIG. 4B; γ, p<0.001, n=5 and δ, p<0.001, compared to control (absence of rosiglitazone or pioglitazone)).

The above data demonstrates that inhibitory effects of TZDs on aromatase do not involve transcription or translation of aromatase gene, but rather interference with substrate (androgen) binding to aromatase.

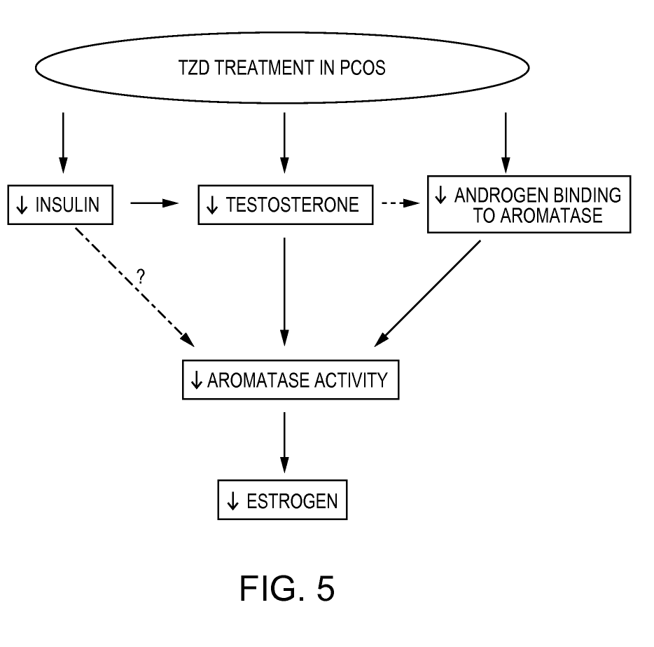

The degree of the TZD-induced inhibition of the estrogen production is compatible to effects of TZDs on the binding of androgen to the aromatase enzyme, thus suggesting that most of the inhibitory effect of TZD on estrogen synthesis is due to the interference of androgen binding to aromatase. In vivo, additional direct inhibitory effect of TZDs on androgen synthesis as well as reduction of hyperinsulinemia due to systemic insulin-sensitizing action of TZDs (with resultant further reduction of androgen synthesis) may produce further decline in estrogen production due to a decrease in available aromatase substrate (FIG. 5).

Effects of TZDs on the Kₘ and Vₘₐₓ of Aromatase in the Presence of Androgens

Granulosa cells were purified on Percoll gradients and cultured as previously described. Purified granulosa cells were resuspended in M199 medium supplemented with 10% heat-inactivated fetal bovine serum (FBS), 10 μg/ml gentamicin and 250 ng/ml amphotericin (Invitrogen Corp., Carlsbad, Calif.). 1 ml of 0.5×10⁵ cells/ml suspension was used the kinetic experiments.

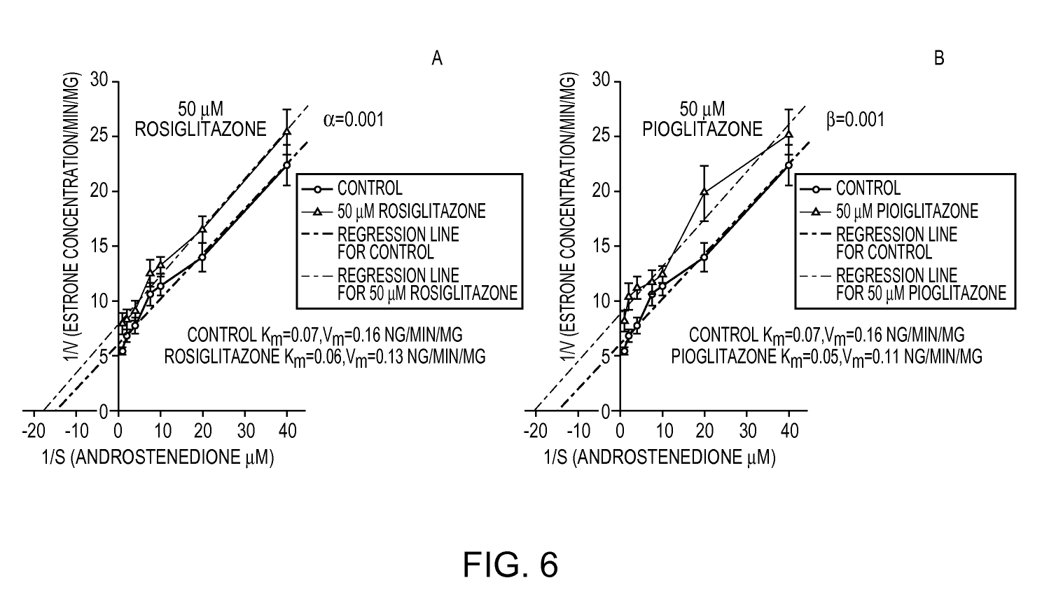

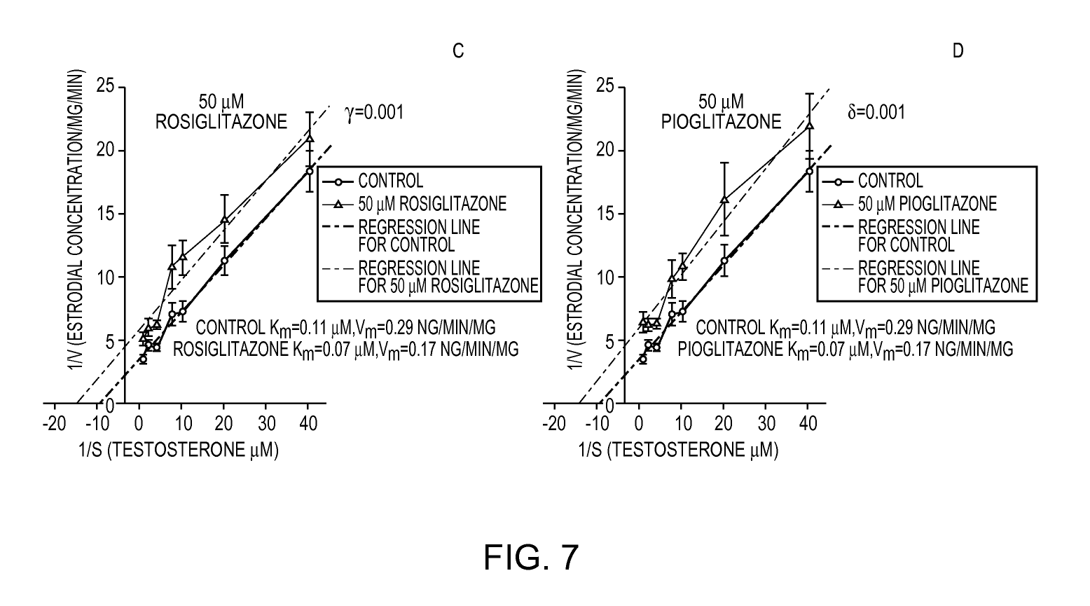

The cells were incubated for 48 h in 5% carbon dioxide and 90% humidity and then for additional 24 h in the same medium containing 2% FBS. The cells were incubated with various concentrations or androstenedione or testosterone (0.025, 0.05, 0.1, 0.133, 0.25, 0.5 or 1.0 μM) with or without rosiglitazone or pioglitazone (50 μM) for 150 min, and the tissue culture medium was collected for determination of estrone and estradiol concentrations. The protein concentrations were determined by modification of Lowry protein assay (Thermo Scientific, Rockford, LI). As shown in FIGS. 6(A&B), when androstenedione was used as the substrate, the Vₘₐₓ of aromatase was reduced by 19% and by 31% and the Kₘ was reduced by 14% and by 20% in the presence of rosiglitazone or pioglitazone, respectively. Similarly, when testosterone was used as the substrate in FIGS. 7 (C & D), both rosiglitazone and pioglitazone inhibited the Vₘₐₓ of aromatase by 41% (p<0.001) and the Kₘ by 36%.

It can be concluded that, in human granulosa cells, TZDs inhibit estrone and estradiol production by interfering with androstenedione or testosterone binding to the aromatase. Therapeutic applications of the partial reduction in binding can be useful in developing new therapies for such diverse disease as PCOS and breast cancer. Given the relative mild effect of TZDs on bone loss, it may be safe to administer TZDs for significant period of time, more than three to five years. When no significant side effects are apparent, TZDs can be administered to partially and reversibly inhibit or interfere with the binding of androgens to aromatase over the remaining lifetime of the patient. Although, the effect of TZDs on bone density has previously been attributed to the adipogenic effect described above, according to the present invention, at least a portion of the decrease in bone density can be due to the inhibition of aromatase.

We claim:

1. A method for the treatment or prophylaxis of an estrogen-dependent disorder in a patient, comprising partially inhibiting the binding of an androgen to aromatase in a patient.

2. A method for the treatment or prophylaxis of an estrogen-dependent disorder in a patient, comprising partially inhibiting the binding of an androgen to aromatase by administering a therapeutically effective amount of a thiazolidinedione to the patient.

3. The method according to claim 2, wherein the therapeutically effective amount of a thiazolidinedione is 2.0 mg to 8.0 mg of rosiglitazone on a daily basis.

4. The method according to claim 2, wherein the therapeutically effective amount of a thiazolidinedione is 5.0 mg to 45.0 mg of pioglitazone on a daily basis.

5. The method according to claim 2, wherein the binding of androgen to aromatase is inhibited at least 20%.

6. The method according to claim 2, wherein the binding of androgen to aromatase is inhibited from about 20% to about 38%.

7. The method according to claim 2, wherein the treatment or prophylaxis lasts longer than three years.

8. The method according to claim 2, wherein the treatment or prophylaxis lasts longer than five years.

9. The method according to claim 2, wherein the patient is a non-human animal.

10. The method according to claim 9, wherein the non-human animal is a mammal.

11. A use of a thiazolidinedione to partially and reversibly inhibit the binding of androgens to aromatase.

12. The use of a thiazolidinedione according to claim 11, wherein the thiazolidinedione is a rosiglitazone.

13. The use of a thiazolidinedione according to claim 11, wherein the thiazolidinedione is a pioglitazone.

14. The use of a thiazolidinedione according to claim 11 to prevent an estrogen-dependent malignancy.

15. The use of a thiazolidinedione according to claim 11 to prevent the recurrence of an estrogen-dependent malignancy.

16. The use of a thiazolidinedione according to claim 11 to treat hyperestrogenism in a patient while reducing estrogen production in that patient by no more than 50 percent.

17. The use of a thiazolidinedione according to claim 11 to treat gynecomastia.

18. The use of a thiazolidinedione according to claim 11 to treat endometriosis.

19. The use of a thiazolidinedione according to claim 11 to treat male infertility.

20. The use of a thiazolidinedione according to claim 11 to treat uterine fibroids.

21. A method of inhibiting aromatase activity in a patient by no more than 50%, comprising administering a therapeutically effective amount of a thiazolidinedione to the patient.

22. A method for the treatment or prophylaxis of a disorder in which estrogen affects the disorder in a patient, comprising administering an amount of a thiazolidinedione sufficient to inhibit the binding of an androgen to aromatase.


SEQUENCE LISTING (2 sequences)

SEQ ID NO: 1 - DNA, 19 bp, Artificial Sequence
  Description of Artificial Sequence Synthetic primer
acccttctgcgtcgtgtca

SEQ ID NO: 2 - DNA, 26 bp, Artificial Sequence
  Description of Artificial Sequence Synthetic primer
gaacttctatggcatctttcaaatcc